(19) United States
(12) Patent Application Publication
(10) Pub. No.: US 2021/0106536 A1
(43) Pub. Date: Apr. 15, 2021

(54) DRUG DELIVERY AGENTS FOR PREVENTION OR TREATMENT OF PULMONARY DISEASE

(71) Applicant: UNIVERSITY INDUSTRY FOUNDATION, YONSEI UNIVERSITY WONJU CAMPUS, Wonju-si (KR)

(72) Inventors: Jaehong KEY, Wonju-si (KR); Sanghyo PARK, Wonju-si (KR)

(21) Appl. No.: 17/108,403

(22) Filed: Dec. 1, 2020

Related U.S. Application Data
(63) Continuation-in-part of application No. PCT/KR2019/006332, filed on May 27, 2019.

(30) Foreign Application Priority Data
Jun. 1, 2018 (KR) 10-2018-0063486

(51) Int. Cl. A61K 9/50; A61K 49/18

(52) U.S. Cl. CPC A61K 9/5031; B82Y 5/00; A61K 49/1857

(57) ABSTRACT

Provided is a lung disease drug delivery carrier, in which the carrier includes a disc particle made of polylactide-co-glycolide (PLGA), the disc particle contains therein a drug, the carrier delivers and/or releases the drug therein into a lung, and the disc particle has a size of 1 μm to 5 μm.

### BACKGROUND

### Field

The present disclosure relates to a lung disease drug delivery carrier that accumulates intensively in lung diseases such as lung cancer.

### Description of the Related Art

The lungs have a very large overall surface area. A thickness of the cells constituting the alveolar sac is 0.1 μm to 0.5 μm, and thus is very small. A density of the cells thereof is lower than that of other cells, so that drug absorption thereto is easy. When a drug is delivered through the lungs, the speed of the systemic circulation of the drug is fast and the drug is not subjected to the first pass metabolism. Thus, the delivery through the lung is very suitable as an administration route of immediate-release drug formulations and has been known as an effective route for local diseases such as asthma/chronic bronchial obstruction.

Further, due to the above characteristics, lung cells exhibit high membrane permeability to macromolecules, and the amount of bioenzymes present in the lung mucosa is relatively small. Thus, the delivery of the drug through the lung is known as effective injection-dependent protein and peptide drug delivery route in the body. In fact, in various literatures, it has been reported that the maximum time to reach the maximum blood concentration of these drugs is about 30 minutes and the bioavailability of these drugs reaches 50% (Leuprolide) compared to the subcutaneous route. In addition, research on drug delivery systems and delivery media through the respiratory tract is actively being conducted due to the improvement of patient convenience that they may take medication on their own.

In the meantime, the drug delivery system refers to a generic term of a series of technologies that control the delivery and release of substances with pharmacological activity to cells, tissues, and organs to exert optimal effects using various physicochemical technologies. The drug delivery system refers to a technology that optimizes drug treatment by designing a formulation to minimize side effects of existing drugs, maximize efficacy and effect, and efficiently deliver the required amount of drug.

Drug delivery systems may be classified based on the route of administration, the type of delivery technology, and the type of drug. Classification based on route of administration may generally include oral, injection, pulmonary inhalation, transdermal, implantation, etc. The classification based on the type of delivery technology may include absorption promotion type, drug effect sustaining type, target site concentration type, and Intelligent DDS.

A carrier for delivering a drug in the drug delivery system as described above may include microparticles or microspheres. It is important to design a formulation to reduce the side effects of the drug, increase patient compliance with the drug, and maximize the efficacy and effect of the drug by efficiently delivering the drug for disease treatment to the treatment site using these drug carriers.

Particularly, the microparticles for drug delivery using biodegradable polymers should be able to easily contain fat-soluble or water-soluble bioactive substances in the microparticles. The microparticles for drug delivery using biodegradable polymers should have properties that the microparticle may contain drugs in the human body and maintain the drugs therein for a certain period of time, safety in decomposition thereof into substances harmless to the human body, and persistence that the microparticle does not release the drug at the initial stage of being injected into the human body, but must have to release the drug for the desired period after reaching the target point.

International Publication No. WO 2015/176025 discloses a non-spherical nano/fine particle and a preparation method thereof used for diagnosis of cancer treatment. In the above patent, a method of delivering a drug including a contrast agent and a therapeutic agent to cells and/or tissues of the body using non-spherical nano/fine particles is known.

### SUMMARY

A purpose of the present disclosure is to provide a lung disease drug delivery carrier that intensively accumulates in lung diseases such as lung cancer.

However, the technical purpose to be achieved by examples of the present disclosure may not be limited to the technical purposes as described above. Other technical challenges may be present.

As a technical means for addressing the above technical problem, a first aspect of the present disclosure provides a lung disease drug delivery carrier in which a drug is introduced into a disc particle including polylactide-co-glycolide (PLGA), wherein the carrier delivers and/or releases the drug therein into the lung, wherein the disc particle is 1 μm to 5 μm in size.

According to one implementation of the present disclosure, the disc particle may have a size of 3 μm, but may not be limited thereto.

According to one implementation of the present disclosure, the drug may include a drug selected from the group consisting of therapeutic agents, contrast agents, diagnostic agents, and combinations thereof, but may not be limited thereto.

According to one implementation of the present disclosure, the disc particle may be decomposed after 24 hours, but may not be limited thereto.

According to one implementation of the present disclosure, the disc particle may further include a polymer selected from the group consisting of polyglycolic acid (PGA), polylactide (PLA), polyglycolide (PG), polyphosphazene, polyiminocarbonate, polyphosphoester, polyanhydride, polyorthoester, and combinations thereof, but may not be limited thereto.

According to one implementation of the present disclosure, the therapeutic agent may include one selected from the group consisting of chemotherapeutic compounds, anti-inflammatory agents, anticancer agents, and combinations thereof, but may not be limited thereto.

According to one implementation of the present disclosure, the therapeutic agents may include, but may not be limited to, those selected from the group consisting of cytotoxic agents, cell arresters, alkylating agents, metabolic antagonists, anti-tumor antibiotics, DNA polymerase inhibitors, DNA gyrase inhibitors, topoisomerase inhibitors, mitosis inhibitors, corticosteroids, intercalating agents, antibodies, hormones, antagonists, and combinations thereof.

According to one implementation of the present disclosure, the chemotherapeutic compounds may include those selected from the group consisting of doxorubicin, vinblastine, vincristine, fludarabine, carmustine, asparaginase, fluorouracil, methotrexate, cyclophosphamide, carboplatin, bleomycin, daunorubicin, lomustine, irinotecan, paclitaxel, docetaxel, etoposide, gemcitabine, imatinib, flutamide, hydroxyurea, trastuzumab, curcumin, temozolomide, and combinations thereof but may not be limited thereto.

According to one implementation of the present disclosure, the drug may include an isotope for nuclear imaging or radiotherapy, but may not be limited thereto.

According to one implementation of the present disclosure, the isotope may include one selected from the group consisting of ⁸⁹Zr, ⁶⁴Cu, ⁶⁸Ga, ⁹⁰Y, ¹⁷⁷Lu, and combinations thereof, but may not be limited thereto.

According to one implementation of the present disclosure, the nuclear imaging may include positron emission tomography (PET), but may not be limited thereto.

According to one implementation of the present disclosure, the contrast agent may include one selected from the group consisting of USPIO, SPIO, Gd chelate, magnetic nanoparticles, and combinations thereof, but may not be limited thereto.

According to one implementation of the present disclosure, the contrast agent may include an optical activator, but may not be limited thereto.

According to one implementation of the present disclosure, the optical activator may include those selected from the group consisting of a green fluorescent protein, a fluorescent chromophore, a chromophore, fluorescent dyes, dyes, cyanine, coumarin, anthracene, acridine, oxazine, arylmethine, tetrapyrrole, pyrene, xanthine, fluorescent molecule, Texas red, FITC, maleimide, cyclic imidyl ester, and combinations thereof, but may not be limited thereto.

The above-described means of solving the problems are merely exemplary and should not be construed as limiting the present disclosure. In addition to the above-described examples, additional examples may be derived from the drawings and detailed description.

According to the above-described means of solving the problem of the present disclosure, the lung disease drug delivery carrier according to the present disclosure uses the biodegradable polymer to be decomposed in the body after 24 hours after injection thereto, and is harmless to human body.

The conventional drug carrier prepared in an emulsion manner has a small size and may have a low drug load amount of around 10 wt %. The time for the carrier to stay in the lungs is also short. However, the lung disease drug delivery carrier according to the present disclosure may have a high drug load amount of around 50 wt %. Due to the size and shape of the disc shape, the drug may be delivered effectively to the target site because the carrier stays longer in the lungs of patients with lung disease compared to normal lungs.

Further, when the drug is delivered directly, the amount of accumulation in the lungs as well as the surrounding organs is large. However, the lung disease drug delivery carrier according to the present disclosure is intensively accumulated in the lungs and a relatively small amount thereof is accumulated in the normal lung for a short period of time. The intensive diagnosis or treatment of lung disease may be achieved.

The effects according to the present disclosure are not limited to the contents exemplified above, and more various effects are included in the present specification.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a schematic diagram of a disc particle preparation process according to one Example of the present disclosure;

FIG. 2 is a scanning electron microscope image of a silicon mold according to one Example of the present disclosure;

FIG. 3 is (a) a scanning electron microscope image of a disc particle with a width of 1 μm; (b) a scanning electron microscope image of a disc particle containing cyanine according to one Example of the present disclosure; (c) a scanning electron microscope image of a 5 μm width disc particle;

FIG. 4 is a graph of a size distribution of disc particles according to one example of the present disclosure;

FIG. 5 is a graph of distribution of the number of disc particles according to one example of the present disclosure;

FIG. 6 is a graph of the absorbance of a drug of a lung disease drug delivery carrier according to one example of the present disclosure;

FIG. 7 is (a) a table of drug loading amounts of lung disease drug delivery carriers according to one Example of the present disclosure and Comparative Example; (b) a graph of the drug release amount of the lung disease drug delivery carrier according to one Example of the present disclosure;

FIG. 8 is an optical microscope image of the lung disease drug delivery carrier according to one Example of the present disclosure and doxorubicin;

FIG. 9 is an optical microscope image of major organs 3 hours after injecting a lung disease drug delivery carrier according to one Example of the present disclosure and Comparative Example into a lung cancer metastasis model;

FIG. 10 is an optical microscope image of a lung cancer metastasis model injected with a lung disease drug delivery carrier according to one Example of the present disclosure;

FIG. 11 is a computed tomography and positron emission tomography image of a lung cancer metastasis model injected with a lung disease drug delivery carrier according to one Example of the present disclosure; and

FIG. 12 is a positron emission tomography image of a lung cancer metastasis model and a normal model injected with a lung disease drug delivery carrier according to one Example of the present disclosure.

### DETAILED DESCRIPTION OF THE EMBODIMENT

Hereinafter, an example of the present disclosure will be described in detail with reference to the accompanying drawings so that a person having ordinary knowledge in the technical field to which the present disclosure belongs may easily implement the same. However, the present disclosure may be implemented in many different forms and may not be limited to the example described herein. Further, in the drawings, in order to clearly explain the present disclosure, parts irrelevant to the description are omitted. Similar reference numerals are attached to similar parts throughout the specification.

It will be understood that when an element or layer is referred to as being “connected to”, or “coupled to” another element or layer, it may be directly on, connected to, or coupled to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

In addition, it will also be understood that when a first element or layer is referred to as being present “on” a second element or layer, the first element may be disposed directly on the second element or may be disposed indirectly on the second element with a third element or layer being disposed between the first and second elements or layers.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof.

The terms “about”, “substantially”, etc. in the present disclosure are used to indicate inherent preparation and substance related tolerance. This is intended to prevent an unscrupulous infringer to design around accurate or absolute values set forth to aid understanding of the present disclosure. The term “step of ˜” used throughout the present disclosure does not mean “step for ˜”.

Throughout the present disclosure, the term “combination thereof” included in expression of a Makushi form means a mixture or combination of at least one selected from the group consisting of elements as recited in the expression of the Makushi form.

As used herein, the term “and/or” includes any and all combinations of one or more of the associated listed items. Expression such as “at least one of” when preceding a list of elements may modify the entire list of elements and may not modify the individual elements of the list.

Throughout the present disclosure specification, the term “biodegradable” means that “a polymer may be chemically broken down in the body to form non-toxic compounds.” In this connection, the decomposition rate is the same as or different from the drug release rate. With the use of biodegradable polymers, the carrier has the property of interacting with the human body without undesirable subsequent effects.

Hereinafter, a lung disease drug delivery carrier according to the present disclosure will be described in detail with reference to an implementation and Example and drawings. However, the present disclosure may not be limited to these implementations and Examples and drawings.

As a technical means for addressing the above technical problem, a first aspect of the present disclosure provides a lung disease drug delivery carrier in which a drug is introduced into a disc particle including polylactide-co-glycolide (PLGA), wherein the carrier delivers and/or releases the drug therein into the lung, wherein the disc particle is 1 μm to 5 μm in size. For example, the disc particle may have a size of 2 μm to 4 μm, but may not be limited thereto.

According to one implementation of the present disclosure, the disc particle may have a size of 3 μm, but may not be limited thereto. The disc particle has a size of 3 μm and has a shape similar to that of red blood cells. Due to its soft nature, the disc particle may significantly reduce the activation of macrophages.

Polylactide-co-glycolide is a biodegradable polymer that is completely decomposed into lactic acid and glycolic acid in the body, but is completely harmless to the human body as the polymer is released as CO₂ out of the body via body metabolism. Thus, the polylactide-co-glycolide is approved by FDA. Further, polylactide-co-glycolide may be formulated in the form of microspheres together with drugs. This formulation prevents the drug from being denatured or aggregated by the external environment such that its activity is changed. Further, polylactide-co-glycolide as a carrier has sustained release property. Thus, at one administration, the drug may last a long time effect in the body. In addition, polylactide-co-glycolide may control the biodegradation period and drug release. That is, varying the copolymer composition and molecular weight thereof may allow controlling the size of the microspheres as a formulation form as needed, and allow the delivery period of the drug to be varied from several weeks to several months. This sustained-release property also has an adjuvant effect, and thus its application range is broad immunologically. However, although polylactide-co-glycolide has been used as a drug carrier in the prior art, it has not been proven that polylactide-co-glycolide is effective in diagnosing or treating lung disease due to intensive accumulation thereof in lung disease sites.

According to one implementation of the present disclosure, the disc particle may further include a polymer selected from the group consisting of polyglycolic acid (PGA), polylactide (PLA), polyglycolide (PG), polyphosphazene, polyiminocarbonate, polyphosphoester, polyanhydride, polyorthoester, and combinations thereof, preferably may further include polyglycolic acid, but may not be limited thereto.

According to one implementation of the present disclosure, the disc particle may be decomposed after 24 hours, but may not be limited thereto. The disc particle is biodegradable using a biodegradable polymer, that is, polylactide-co-glycolide, and thus has the advantage of being harmless to the human body.

According to one implementation of the present disclosure, the drug may include a drug selected from the group consisting of therapeutic agents, contrast agents, diagnostic agents, and combinations thereof, but may not be limited thereto.

The lung disease drug delivery carrier may contain the drug introduced into the disc particle. The carrier may deliver and/or release the drug to the lung to diagnose, image, or treat the lung disease.

According to one implementation of the present disclosure, the therapeutic agent may include one selected from the group consisting of chemotherapeutic compounds, anti-inflammatory agents, anticancer agents, and combinations thereof, but may not be limited thereto.

According to one implementation of the present disclosure, the therapeutic agents may include, but may not be limited to, those selected from the group consisting of cytotoxic agents, cell arresters, alkylating agents, metabolic antagonists, anti-tumor antibiotics, DNA polymerase inhibitors, DNA gyrase inhibitors, topoisomerase inhibitors, mitosis inhibitors, corticosteroids, intercalating agents, antibodies, hormones, antagonists, and combinations thereof.

According to one implementation of the present disclosure, the chemotherapeutic compounds may include those selected from the group consisting of doxorubicin, vinblastine, vincristine, fludarabine, carmustine, asparaginase, fluorouracil, methotrexate, cyclophosphamide, carboplatin, bleomycin, daunorubicin, lomustine, irinotecan, paclitaxel, docetaxel, etoposide, gemcitabine, imatinib, flutamide, hydroxyurea, trastuzumab, curcumin, temozolomide, and combinations thereof, preferably the chemotherapeutic compounds may include doxorubicin, but may not be limited thereto.

According to one implementation of the present disclosure, the drug may include an isotope for nuclear imaging or radiotherapy, but may not be limited thereto.

According to one implementation of the present disclosure, the isotope may include one selected from the group consisting of ⁸⁹Zr, ⁶⁴Cu, ⁶⁸Ga, ⁹⁰Y, ¹⁷⁷Lu, and combinations thereof, but may not be limited thereto.

According to one implementation of the present disclosure, the nuclear imaging may include positron emission tomography (PET), but may not be limited thereto. The positron emission tomography is one of nuclear medicine testing methods capable of displaying physiological, chemical, and functional images of the human body in three dimensions using radiopharmaceuticals that emit positrons. Currently, PET is mainly used to diagnose various cancers, and is known as a useful test for differential diagnosis, staging, recurrence evaluation, and treatment effect determination for cancer. In addition, positron emission tomography may be used to obtain receptor images or metabolic images for heart disease, brain disease and brain function evaluation.

According to one implementation of the present disclosure, the contrast agent may include one selected from the group consisting of USPIO, SPIO, Gd chelate, magnetic nanoparticles, and combinations thereof, but may not be limited thereto.

According to one implementation of the present disclosure, the contrast agent may include an optical activator, but may not be limited thereto.

According to one implementation of the present disclosure, the optical activator may include those selected from the group consisting of a green fluorescent protein, a fluorescent chromophore, a chromophore, fluorescent dyes, dyes, cyanine, coumarin, anthracene, acridine, oxazine, arylmethine, tetrapyrrole, pyrene, xanthine, fluorescent molecule, Texas red, FITC, maleimide, cyclic imidyl ester, and combinations thereof, but may not be limited thereto. Preferably, the optical activator may include a green fluorescent protein, but may not be limited thereto.

The green fluorescent protein refers to a protein capable of emitting green light in a living body to observe how proteins function in a living body. A gene of a fluorescent protein may be attached to a protein to be tracked and then may be injected into a cell. Thus, the movement, location, and growth process of the protein may be easily identified based on the green fluorescent protein. Green fluorescent protein enables observation of phenomena occurring in the human body that otherwise could not be observed with the eye before, thus tracking the proliferation of neurons, the spread of cancer cells, or the destruction of brain neurons in Alzheimer's patients.

Hereinafter, the present disclosure will be described in more detail based on Examples, but the following Examples is for illustrative purposes only and is not intended to limit the scope of the present disclosure.

### [Example 1] Preparation of Disc Particle

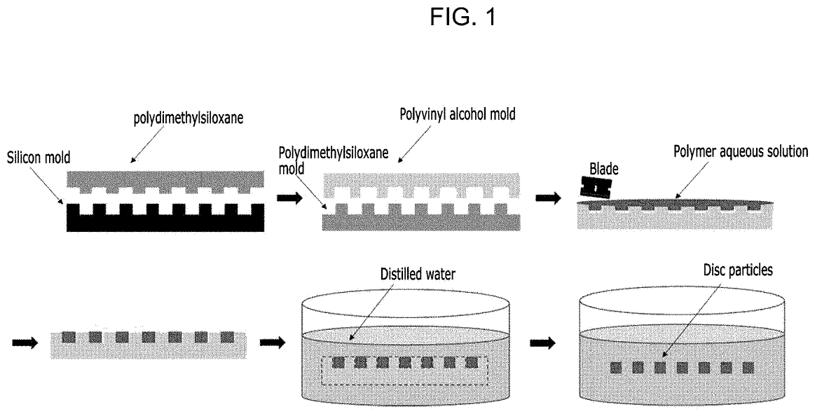

Referring to FIGS. 1 to 3, the method for preparing the disc particle will be described.

FIG. 1 is a schematic diagram of the disc particle preparation process according to one Example of the present disclosure.

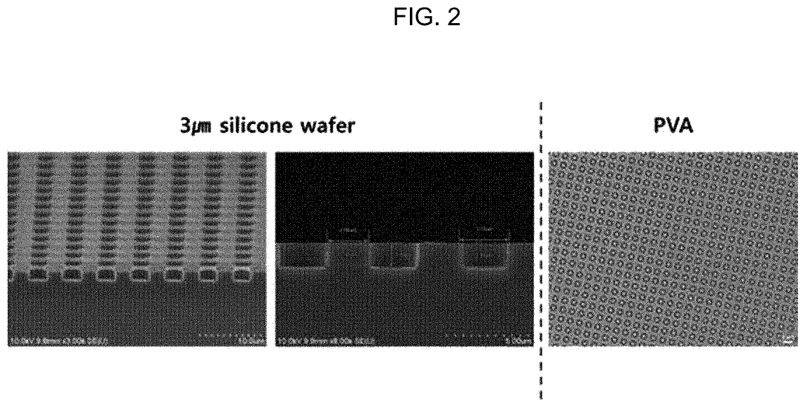

First, a silicon mold having millions of pillars having a width of 3 μm and a depth of 1.5 μm was produced using electron beam lithography. FIG. 2 is a scanning electron microscope image of a silicon mold according to one Example of the present disclosure. Referring to FIG. 2, it may be seen that the silicon mold having millions of pillars having a width of 3 μm and a depth of 1.5 μm is formed. Thereafter, a polydimethylsiloxane layer was deposited on the silicon mold to prepare a polydimethylsiloxane mold having pillars having the same size and shape as that of the silicon mold. Subsequently, a polyvinyl alcohol layer was deposited on the polydimethylsiloxane mold to prepare a polyvinyl alcohol mold having the same pillars as those of the polydimethylsiloxane mold.

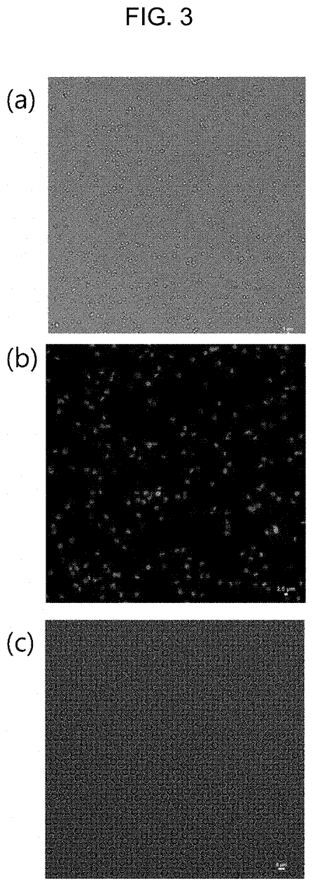

A polymer aqueous solution containing polylactide-co-glycolide was deposited on the polyvinyl alcohol mold and then subjected to polymerization via exposure to UV light. Then, the polyvinyl alcohol mold was dissolved in distilled water, and disc particles having a width of 3 μm were collected via centrifugation. (a) OF FIG. 3 is a scanning electron microscope image of a disc particle with a width of 1 μ:m; (b) OF FIG. 3 is a scanning electron microscope image of a disc particle containing cyanine according to one Example of the present disclosure; (c) OF FIG. 3 is a scanning electron microscope image of a 5 μm width disc particle. Referring to (b) OF FIG. 3, it may be identified that the disc particle according to Example 1 has a shape similar to that of red blood cells.

### [Example 2] Preparation of Lung Disease Drug Delivery Carrier

A polymer aqueous solution containing polylactide-co-glycolide and doxorubicin was deposited on the polyvinyl alcohol mold prepared in Example 1 and then subjected to polymerization via exposure to UV light. Subsequently, the polyvinyl alcohol mold was dissolved in distilled water, and a lung disease drug delivery carrier having a width of 3 μm was collected via centrifugation.

### Comparative Example 1

Mesitylene was added to an aqueous solution of CTAB and NaOH. TEOS was added thereto while stirring at 80° C. Then, the mixture was washed with methanol and dried at 65° C. for one day. Crude MSN was added to methanol and HCl aqueous solution, and sonication was performed. Then, CTAB and mesitylene were removed therefrom while stirring at 50° C. MSN was washed with methanol and dried at 65° C. The dried MSN along with APTES was added to a toluene solution and reaction occurred at 110° C. for 15 hours. Thereafter, MSN-NH₂ was washed with ethanol and hexane and then dried at 65° C. for one day. MSN binds to hyaluronic acid via peptide bonds. MSN binding to hyaluronic acid was added to D.I water and then doxorubicin was added thereto while stirring. In this way, mesoporous silica nanoparticles having a self-assembled particle structure were prepared.

### Comparative Example 2

In Comparative Example 2, only the drug was delivered without a separate disc particle. The drug employed the same doxorubicin as in Example 2.

### Experimental Example

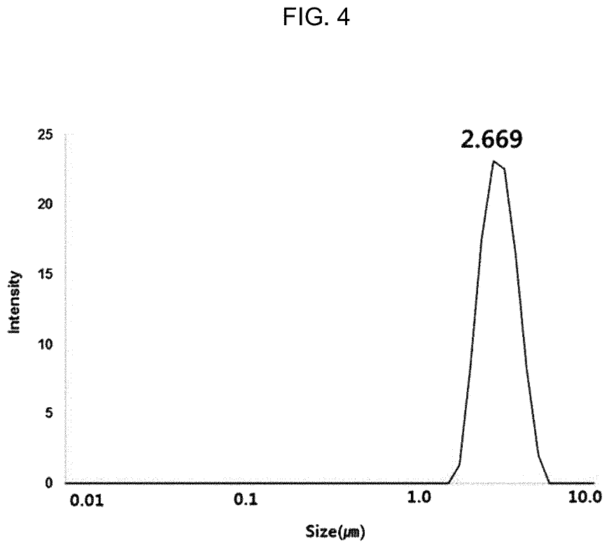

FIG. 4 is a graph of the size distribution of disc particles according to one example of the present disclosure.

Referring to FIG. 4, the disc particle according to one Example of the present disclosure has an average diameter of 2.669 μm.

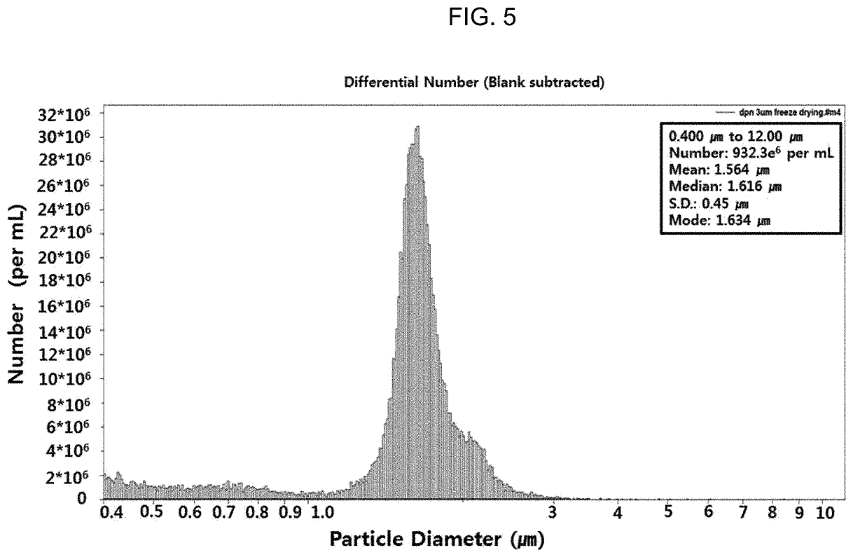

FIG. 5 is a graph of the distribution of the number of disc particles according to one example of the present disclosure.

Referring to FIG. 5, it may be seen that 486.1 e⁶ disc particles are produced per one polyvinyl alcohol mold.

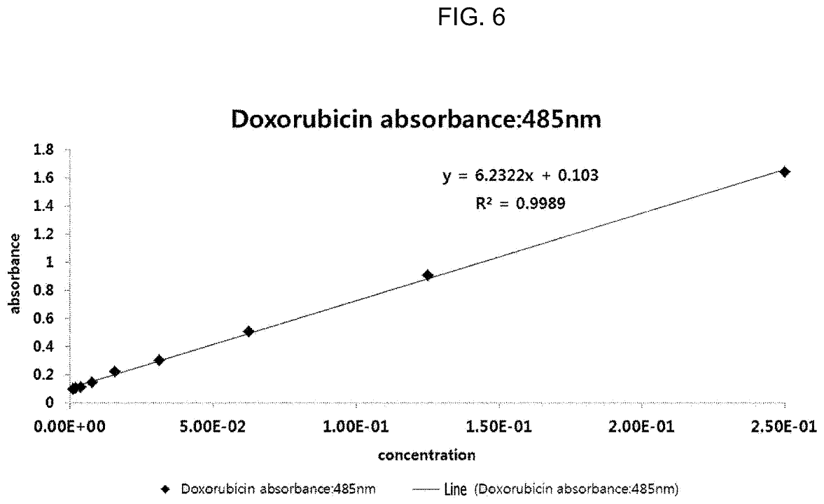

FIG. 6 is a graph of the absorbance of a drug of a lung disease drug delivery carrier according to one example of the present disclosure.

Referring to FIG. 6, the drug loading amount of the lung disease drug delivery carrier according to Example 2 may be identified. Thus, it may be identified that the absorbance of doxorubicin is proportional to the concentration of doxorubicin.

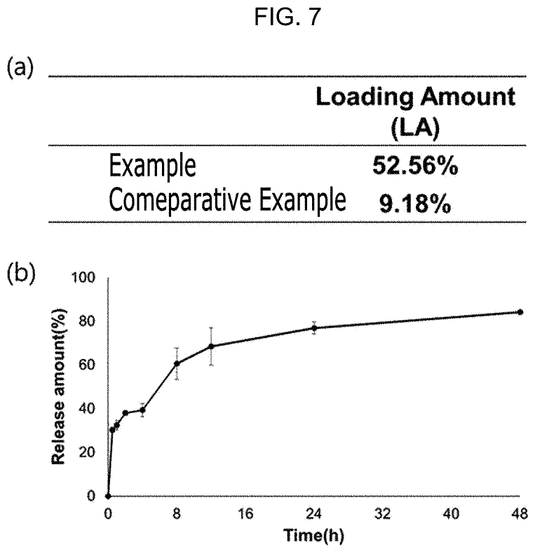

(a) OF FIG. 7 is a table of drug loading amounts of lung disease drug delivery carriers according to one Example of the present disclosure and Comparative Example; (b) OF FIG. 7 is a graph of the drug release amount of the lung disease drug delivery carrier according to one Example of the present disclosure. The drug loading amount may be expressed as (weight of drug contained in disc particle/weight of disc particle)×100.

Referring to (a) OF FIG. 7, it may be identified that the drug loading amount of the drug carrier according to Comparative Example 1 is 9.18%, whereas the drug loading amount of the lung disease drug delivery carrier according to Example 2 is 52.56%, which is about 6 times of the drug loading amount of the drug carrier according to Comparative Example 1.

Referring to (b) OF FIG. 7, it may be identified that the lung disease drug delivery carrier according to Example 2 of the present disclosure releases the drug quickly while initially staying in the lungs for 8 hours and thereafter, releases the drug constantly regardless of time.

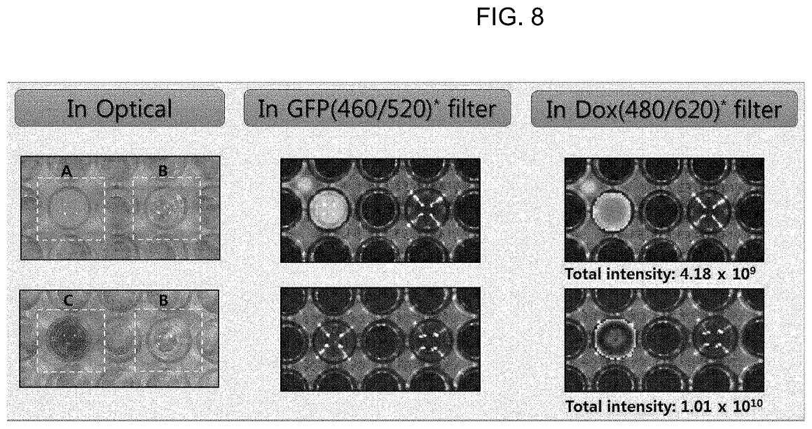

FIG. 8 is an optical microscope image of the lung disease drug delivery carrier and doxorubicin according to one Example of the present disclosure. A is an optical microscope image of a lung disease drug delivery carrier according to Example 2 of the present disclosure as diluted in phosphate buffered physiological saline. B is an optical microscope image containing only phosphate buffered physiological saline. C is an optical microscope image of doxorubicin diluted in phosphate buffered physiological saline. It may be identified that both the disc particle and the lung disease drug delivery carrier exhibit fluorescence only in a Dox filter.

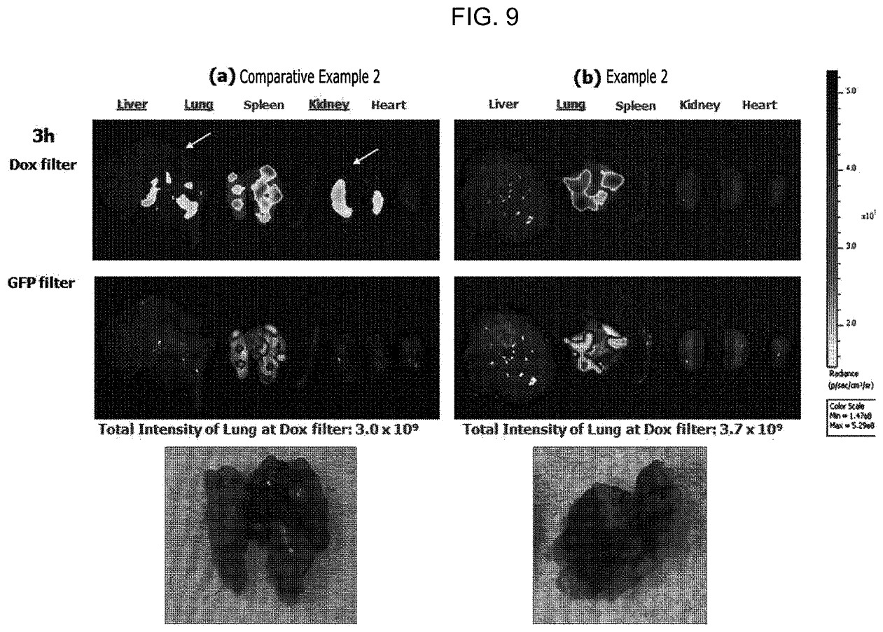

FIG. 9 is an optical microscope image of major organs 3 hours after injecting a lung disease drug delivery carrier according to one Example of the present disclosure and Comparative Example into a lung cancer metastasis model. A GFP filter image may identify the presence or absence of cancer and a location thereof by labeling cancer cells with GFP (green fluorescent protein).

Referring to FIG. 9, it may be identified that the drug according to Comparative Example 2 is accumulated in the liver and kidneys in addition to the lungs of the lung cancer metastasis model, whereas the lung disease drug delivery carrier according to Example 2 is concentrated only in the lung cancer site of the lung cancer metastasis model.

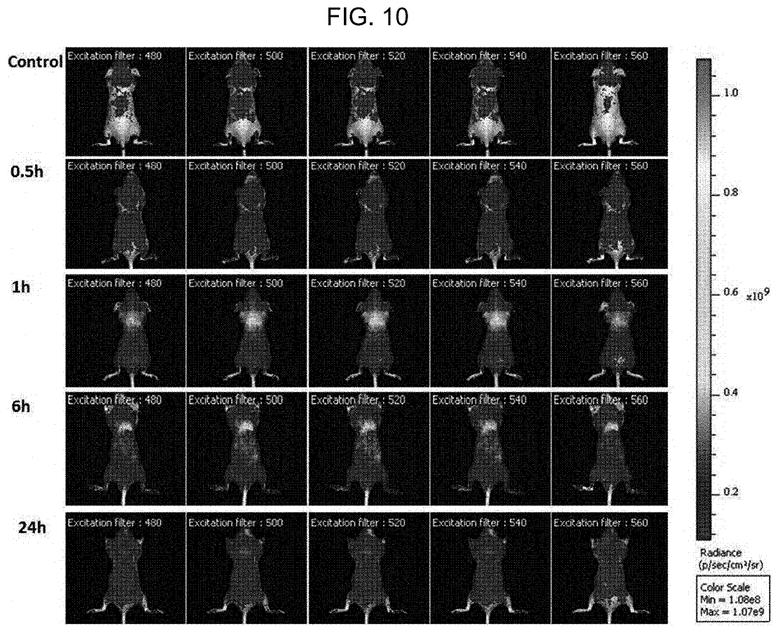

FIG. 10 is an optical microscope image of a lung cancer metastasis model injected with a lung disease drug delivery carrier according to one Example of the present disclosure.

Referring to FIG. 10, it may be identified that lung disease drug delivery carriers according to Example 2 of the present disclosure accumulate in the lungs, then gradually biodegrade, such that most thereof is removed after 6 hours.

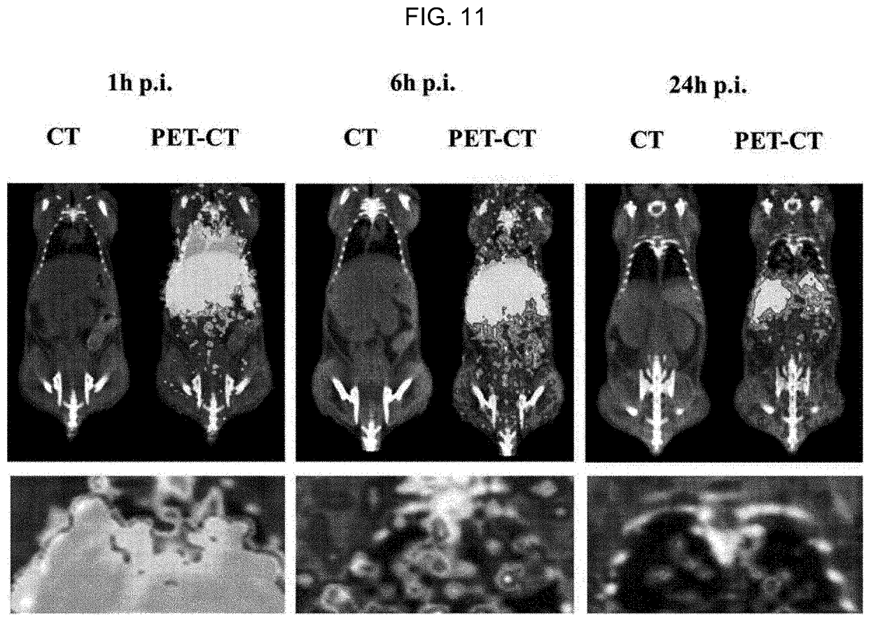

FIG. 11 is a computed tomography and positron emission tomography image of a lung cancer metastasis model injected with a lung disease drug delivery carrier according to one Example of the present disclosure.

Referring to FIG. 11, it may be identified that lung disease drug delivery carriers according to Example 2 of the present disclosure accumulate in the lungs, gradually biodegrade, such that a large amount thereof is removed after 6 hours.

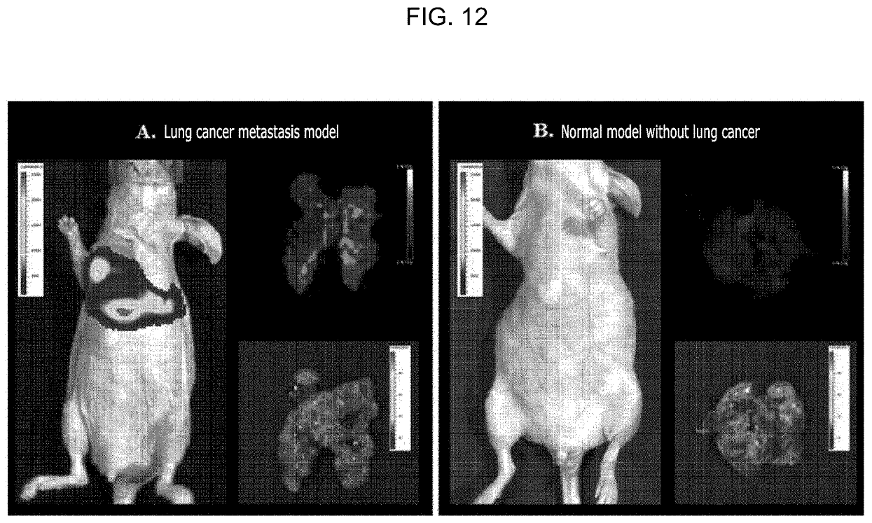

FIG. 12 is a positron emission tomography image of a lung cancer metastasis model and a normal model injected with a lung disease drug delivery carrier according to one Example of the present disclosure.

Referring to FIG. 12, it may be seen that the lung disease drug delivery carrier according to Example 2 of the present disclosure accumulates in a larger amount in the lung cancer metastasis model, compared to the normal model without lung cancer. Thus, using the lung disease drug delivery carrier according to Example 2, lung diseases such as lung cancer may be intensively diagnosed, and may be treated by effectively delivering the drug thereto.

The description of the present disclosure above is for illustration purposes only. Those of ordinary skill in the technical field to which the present disclosure belongs may understand that it is possible to easily vary the present disclosure into other concrete forms without changing the technical idea or essential characteristics of the present disclosure. Therefore, the examples described above are illustrative in all respects and should be understood as non-limiting. For example, components described in a single form may be implemented in a distributed manner. Likewise, components that are described as being distributed may be implemented in a combined form.

The scope of the present disclosure is indicated by the claims to be described later rather than the detailed description. The meaning and scope of the claims and all changes or modifications derived from the concept of equivalent should be construed as being included in the scope of the present disclosure.

Although the examples of the present disclosure have been described in detail with reference to the accompanying drawings, the present disclosure may not be limited thereto and may be embodied in many different forms without departing from the technical concept of the present disclosure. Therefore, the examples of the present disclosure are provided for illustrative purposes only but not intended to limit the technical spirit of the present disclosure. The scope of the technical spirit of the present disclosure may not be limited thereto. Therefore, it should be understood that the above-described examples are illustrative in all aspects and do not limit the present disclosure. The protective scope of the present disclosure should be construed based on the following claims, and all the technical concepts in the equivalent scope thereof should be construed as falling within the scope of the present disclosure.

What is claimed is:

1. A lung disease drug delivery carrier, wherein the carrier includes a disc particle made of polylactide-co-glycolide (PLGA), wherein the disc particle contains therein a drug, wherein the carrier delivers and/or releases the drug therein into a lung, wherein the disc particle has a size of 1 μm to 5 μm.

2. The lung disease drug delivery carrier of claim 1, wherein the disc particle has a size of 3 μm.

3. The lung disease drug delivery carrier of claim 1, wherein the drug includes one selected from a group consisting of a therapeutic agent, a contrast agent, a diagnostic agent, and combinations thereof.

4. The lung disease drug delivery carrier of claim 1, wherein the disc particle is decomposed after 24 hours after being injected to a body.

5. The lung disease drug delivery carrier of claim 1, wherein the disc particle further includes a polymer selected from a group consisting of polyglycolic acid (PGA), polylactide (PLA), polyglycolide (PG), polyphosphazene, polyiminocarbonate, polyphosphoester, polyanhydride, polyorthoester, and combinations thereof.

6. The lung disease drug delivery carrier of claim 3, wherein the therapeutic agent includes one selected from a group consisting of a chemotherapeutic compound, an anti-inflammatory agent, an anticancer agent, and combinations thereof.

7. The lung disease drug delivery carrier of claim 3, wherein the therapeutic agent includes one selected from a group consisting of a cytotoxic agent, a cell arrester, an alkylating agent, a metabolic antagonist, an anti-tumor antibiotic, a DNA polymerase inhibitor, a DNA gyrase inhibitor, a topoisomerase inhibitor, a mitosis inhibitor, corticosteroid, an intercalating agent, an antibody, hormone, antagonist, and combinations thereof.

8. The lung disease drug delivery carrier of claim 6, wherein the chemotherapeutic compound includes one selected from a group consisting of doxorubicin, vinblastine, vincristine, fludarabine, carmustine, asparaginase, fluorouracil, methotrexate, cyclophosphamide, carboplatin, bleomycin, daunorubicin, lomustine, irinotecan, paclitaxel, docetaxel, etoposide, gemcitabine, imatinib, flutamide, hydroxyurea, trastuzumab, curcumin, temozolomide, and combinations thereof.

9. The lung disease drug delivery carrier of claim 3, wherein the drug includes an isotope for nuclear imaging or radiotherapy.

10. The lung disease drug delivery carrier of claim 9, wherein the isotope includes one selected from a group consisting of ⁸⁹Zr, ⁶⁴Cu, ⁶⁸Ga, ⁹⁰Y, ¹⁷⁷Lu, and combinations thereof.

11. The lung disease drug delivery carrier of claim 9, wherein the nuclear imaging includes positron emission tomography (PET).

12. The lung disease drug delivery carrier of claim 3, wherein the contrast agent includes one selected from a group consisting of USPIO, SPIO, Gd chelate, magnetic nanoparticles, and combinations thereof.

13. The lung disease drug delivery carrier of claim 3, wherein the contrast agent includes an optical activator.

14. The lung disease drug delivery carrier of claim 13, wherein the optical activator includes one selected from a group consisting of a green fluorescent protein, a fluorescent chromophore, a chromophore, fluorescent dyes, dyes, cyanine, coumarin, anthracene, acridine, oxazine, arylmethine, tetrapyrrole, pyrene, xanthine, fluorescent molecule, Texas red, FITC, maleimide, cyclic imidyl ester, and combinations thereof.
